(19) United States
(12) Patent Application Publication
(10) Pub. No.: US 2012/0149056 A1
(43) Pub. Date: Jun. 14, 2012

(54) METHOD AND REAGENTS FOR DETECTING THE PRESENCE OR ABSENCE OF A TARGET SUBSTANCE IN A TEST SAMPLE

(75) Inventor: Stephen C. Edberg, Longboat Key, FL (US)

(73) Assignee: PILOTS POINT LLC, Sarasota, FL (US)

(21) Appl. No.: 12/823,698

(22) Filed: Jun. 25, 2010

Related U.S. Application Data
(60) Provisional application No. 61/269,587, filed on Jun. 27, 2009.

(51) Int. Cl. C12Q 1/04

(52) U.S. Cl. 435/34

(57) ABSTRACT

Reagent mixtures and testing procedures for detecting the presence or absence of various target substances such as aureus; vancomycin-resistant enterrococcus; Group B Streptococcus; E. coli; fungi; and others, involve the use of a testing reagents which include growth factors for the particular target substance, a detectable signal generator which is sensitive to the presence of the target substance, and hatmonal compounds which will accelerate the growth of the target substance within a specimen being tested. Examples of specimen samples that can be tested include nasal swabs, throat swabs, lesion swabs, environmental swabs, foodstuffs, and water, to mention a few.

This application claims the benefit of U.S. Ser. No. 61/269,587 filed Jun. 27, 2009.

### BACKGROUND OF THE INVENTION

1. Technical Information

The present method and test mixture relates to the detection of target bacteria in a biological, environmental, or food sample, and more particularly to those methods and test mixtures utilizing reacting factors which include hormones which enhance growth of the target bacteria.

2. Background Information

One bacterium which can be detected is Staphylococcus aureus (S. aureus) which can be a virulent pathogen of animals and humans. Moreover, it can cause severe food poisoning by the production of a toxin. Diseases caused by S. aureus cover a very wide clinical spectrum, from simple skin infections to life threatening infections of the bones, heart, and organs. Of particular concern is the recognition that S. aureus infection is common after surgery. It is also associated with intravenous tubing and other implants.

The bacterium S. aureus may be transmitted between healthy individuals by skin to skin contact, or from a commonly shared item or a surface (e.g., tanning beds, gym equipment, food handling equipment, etc.) where the transfer may be made to a subsequent person who uses the shared item or touches the surface. Of great medical concern is the recognition that healthy people entering hospitals may “carry” S. aureus (e.g., on their skin, or in their noses, etc.) without any signs or symptoms that they do so. In the presence of favorable conditions (often found in but not limited to hospitals), the S. aureus can activate and cause serious infection. In addition, S. aureus can also be a source of food poisoning, often caused by a food handler contaminating the food product (e.g., meat, poultry, eggs, salads containing mayonnaise, bakery products, dairy products, etc.).

There are two categories of S. aureus based on an individual clone's susceptibility to the class of antibiotics that began with methicillin. These are methicillin susceptible S. aureus (MSSA), and methicillin resistant S. aureus (MRSA). Until only a few years ago, MRSA was most commonly found in hospitals. Now, many are also present in the noses, skin, etc. of people in the non-hospital community. Moreover, these MRSA are increasingly causing serious infections in the community. MRSA is particularly serious because very few antibiotics (e.g., vancomycin) have been shown to be uniformly effective against MRSA.

The Center for Disease Control and Prevention actively surveys for the development of methicillin resistant S. aureus. In 2000, the Society for Healthcare Epidemiology of America guidelines recommended contact isolation for patients with MRSA. In addition to the morbidity and mortality caused by MRSA, it has been estimated that each case of infection costs at least $23,000. Accordingly, many hospitals and nursing homes proactively sample patients for MRSA [Clany, M., Active Screening in High-Risk units is an effective and cost-avoidant method to reduce the rate of methicillin-resistant Staphylococcus aureus infection in the hospital, Infection Control and Hospital Epidemiology 27:1009-1017, 2006].

Another lethal pathogen that is found in biological and environmental environs is E. coli. This pathogen is found most often in water and in food stuffs that have been infected with it. U.S. Pat. No. 4,925,789 granted May 15, 1990 to Stephen C. Edberg is incorporated herein by reference and describes a method and formulation used to detect E. coli in water or in other test samples. The subject matter of this patent is found in a commercial product marketed under the registered trademark COLILERT. The COLILERT product and procedure can typically detect E. coli in a sample being tested in 8-24 hours after initializing the test procedure.

It would be desirable to provide a test mixture and a method that can rapidly detect MSSA, MRSA, E. coli, or other bacterial pathogens, directly from a first generation sample; one that does not require a skilled technician to perform the method; one that can be performed without the need to develop isolates from the specimen sample (i.e., one that can be performed on a “first generation” specimen sample); and one that does not require a large concentration of the target organisms to be accurate.

### SUMMARY OF THE INVENTION

This invention relates to a method and test mixture for specific detection of a target bacteria, such as S. aureus bacteria, E. coli, fungi, or the like in a biological, environmental, or food specimen. The test mixture will include suitable plant growth hormone components including, but not limited to, indoleacedic acid, gibberellic acid and kinetin. These ingredients speed up the test so as to arrive at a definitive result sooner than if the plant growth hormone ingredients were not included in the testing mixture.

According to an aspect of the present invention, a method for detecting a target substance in a specimen sample is provided. The method includes the steps of: a) providing a medium for use in detecting the target substance which medium includes target substance growth-promoting components which are selectively operative to stimulate growth of the target substance when combined therewith, said components including hormonal components which will enhance growth of the target substance, and said medium further including one or more components that will produce a detectable signal in the presence of the target substance; b) combining the medium with the specimen sample; and c) incubating the specimen sample in the medium for a time sufficient to produce the detectable signal in the event that the specimen contains the target substance.

According to another aspect of the present invention, a medium for use in detecting a target bacteria is provided. The medium includes effective amounts of target bacteria growth-promoting components which are selectively operative to stimulate growth of the target bacteria when combined therewith. The components include effective amounts of hormonal components which will enhance growth of the target bacteria. The medium further includes effective amounts of one or more components that will produce a detectable signal in the presence of the target bacteria.

### DETAILED DESCRIPTION

The method and test reagents include components which will provide selective growth of the target bacteria in a mixture of the reagents and the sample being tested. The reagents will also include a component or components that will produce a detectable signal in the reagents and sample in the event that the target is present in the sample. Such components can take the form of target-specific antigens or nucleic acids, to mention a few. The test mixture will also include suitable amounts of plant growth hormone components such as indoleacedic acid, gibberellic acid and kinetin, for example.

The test reagents are preferably prepared in a faun that facilitates handling, packaging, storing, etc., of the test reagents. A dry powder that can be hydrated into liquid form is a particularly preferable form for the test reagents, but the present invention is not limited to a powder form. The test reagents may assume a liquid form, or any other form (e.g., paste, gel, etc.), preferably one that can be hydrated for use. The hormonal ingredient or ingredients will be included in the test reagents, whatever form they take.

Effective hydrated test mixtures for testing for S. aureus can be made using the following constituents in the ranges indicated, to create 15 ml of test mixture:

| | Quantity per 15 ml | | Range per 15 ml |
|---|---|---|---|
| Constituent | of Test Mixture | | of Test Mixture |
| Nitrate Broth | 7.5 | ml | 1.0 ml-9.0 ml |
| Water | 7.5 | ml | 1.0 ml-9.0 ml |
| Uracil | 10.0 | mg | 1.0 mg-20.0 mg |
| Sodium Pyruvate | 10.0 | mg | 1.0 mg-20.0 mg |
| L-glutamine | 20.0 | mg | 5.0 mg-40.0 mg |
| Sodium Sulfite | 1.0 | mg | 0.5 mg-2.0 mg |
| Rabbit Plasma | 100.0 | mg | 15.0 mg-500.0 mg |
| Fibrinogen | 100.0 | mg | 15.0 mg-500.0 mg |
| Gentamicin* | 15.0 | mcg | 10.0 mcg-20.0 mcg |
| Hydrolyzable | 500.0 | mg | 200.0 gm-800.0 mg |
| Substrate* | | | |
| Sugars* | 15.0 | gm | 10.0 gm-25.0 gm |
| Kinetin | 0.001 | gm/L | 0.0001 gm/L-0.01 gm/L |
| Indole Butyric Acid** | 0.005 | gm/L | 0.005 gm/L-0.05 gm/L |
| Gibberellic Acid | 0.001 | gm/L | 0.001 gm/L-0.01 gm/L |

*Optional for MRSA detection:
**Indoleacetic Acid can be substituted

In addition to the hormonal components, additional growth promoting constituents will be included within the test reagents so as to facilitate the multiplication and sustaining of the target bacteria. The constituents can be varied to suit the application. Those in the art will recognize that many different combinations of constituents, and varying relative amounts of the constituents, can be used to provide the same functionality Growth promoting constituents include sources of nitrates and proteins, material operative to assist in the generation of nucleic acid synthesis, sources of energy for the target bacteria, sources of amino acid growth factor, and in some embodiments materials operable to help repair damaged target organisms. This list of growth promoting constituents does not represent all of the materials that can be beneficial within the test reagents, but does illustrate materials that are acceptable (e.g., vitamins, salts, minerals, inorganic moieties, etc.). The test reagents may include other constituents that benefit the performance of the test reagents.

In most applications of the present invention, it will be desirable to utilize test reagents that include the following: a) an effective amount of amino acids; b) an effective amount of nitrogen sources; c) an effective amount of salts; d) an effective amount of vitamins; and e) an effective amount of calcium. Those skilled in the art will recognize that natural sources of such amino acids can be used rather than pure sources. The natural sources (e.g. extract of whole organisms, such as yeast) may be in mixture form or in purified form. The natural mixtures can contain varying amounts of such amino acids and vitamins. Those skilled in the art will further recognize that many different combinations of amino acids and vitamins can be used in present invention test reagents.

Those skilled in the art will further recognize that carbon, nitrogen, trace elements, vitamins, amino acids and selective agents can be provided in many forms. Generally, it is preferred to have an amount of vitamins and amino acids within a predetermined range, but those in the art will recognize that the actual properties of each ingredient may be varied so that reduction in the amount of one ingredient can be compensated by an increase in the amount of another. This is particularly relevant when the essential amino acids, trace elements or vitamins of the microbes sought to be detected are known. Some ingredients may be provided in reduced amounts or deleted if they may be synthesized endogenously by the microorganism whose presence is to be determined. Salts may be provided as a source of ions upon dissociation. The hormonal ingredient or ingredients will be included in effective amounts in the test reagents.

The test reagents may be packaged in a container (e.g., a test tube, a container with a flat bottom wall, etc.) that facilitates the testing process. If the medium is prepared in a form that can be hydrated, the reagents can be hydrated with sterile water or non-sterile water.

To detect the presence of a target bacteria within a sample, the sample is obtained from a biological, environmental, or food specimen. A sample collected using a sample swab is an example of a first generation sample that is particularly convenient to collect and test using the present invention. Once collected, the sample is inoculated into the test mixture.

The inoculated sample may be incubated under conditions favorable to facilitate the multiplication of any target bacteria that may be present within the inoculated sample. In the case of a powdered test mixture hydrated with water, the incubation may be carried out at temperatures between about 200° C. to 42° C., which may vary with the target. The combination of sequential enzyme specificity, target bacteria enhancing growth factors, and, in certain cases, antibiotic selectivity, provides multiple hurdles which prevent the competing non-target bacteria from being detected within the test period. The inclusion of the hormonal compounds in the reagents will enhance and speed up the analysis.

The present invention test and method can be used in hospital admissions, routinely in intensive care units, in nursing homes, dialysis patients, people receiving home immunosuppressive therapy, and the like. It can also be used in environmental settings (e.g., gyms, tanning salons, restaurants, water etc.) where the target bacteria may be found.

The hormonal ingredient or ingredients can be used in other bacteria tests to improve the production of an end result. Such other tests can include a vancomycin-resistant enterococcus test; a Group B Streptococcus test; a test for heomolytic E. coli; and a test for Listeria monocytogenes, to name a few.

While the invention has been described with respect to preferred embodiments, those skilled in the art will readily appreciate that various changes and/or modifications can be made to the invention without departing from the spirit or scope of the invention as defined by appended claims.

What is claimed is:

1. A method for detecting a target substance in a specimen sample, said method comprising the steps of:
a) providing a medium for use in detecting the target substance which medium includes target substance growth-promoting components which are selectively operative to stimulate growth of the target substance when combined therewith, said components including hormonal components which will enhance growth of the target substance, and said medium further including one or more components that will produce a detectable signal in the presence of the target substance;
b) combining said medium with said specimen sample; and
c) incubating said specimen sample in said medium for a time sufficient to produce said detectable signal in the event that said specimen contains said target substance.

2. The method of claim 1 wherein said hormonal components are plant hormones.

3. The method of claim 1 wherein said hormonal components include at least one component selected from the group consisting of kinetin, indoleacedic acid, indole butyric acid and gibberellic acid.

4. A medium for use in detecting a target bacteria, said medium including effective amounts of target bacteria growth-promoting components which are selectively operative to stimulate growth of the target bacteria when combined therewith, and said components including effective amounts of hormonal components which will enhance growth of the target bacteria, and said medium further including effective amounts of one or more components that will produce a detectable signal in the presence of the target bacteria.

5. The medium of claim 4 wherein said hormonal components are plant hormones.

6. The medium of claim 4 wherein said hormonal components include at least one component selected from the group consisting of kinetin, indole acetic acid, indole butyric acid and gibberellic acid.
